(19) United States
(12) Patent Application Publication
(10) Pub. No.: US 2024/0165109 A1
(43) Pub. Date: May 23, 2024

(54) Methods of Treatment with Selective CB2 Receptor Agonists

(71) Applicant: Arena Pharmaceuticals, Inc., New York, NY (US)

(72) Inventors: Fabio Cataldi, Beverly, MA (US); Brett Alan English, Brentwood, TN (US); Beatriz Fioravanti Lindstrom, San Diego, CA (US); Charlies Chunhua Liu, Irvine, CA (US); Sharon Diane Skare, San Diego, CA (US); Stewart Alleyn Turner, Thousand Oaks, CA (US)

(73) Assignee: Arena Pharmaceuticals, Inc., New York, NY (US)

(21) Appl. No.: 18/548,622

(22) PCT Filed: Mar. 1, 2022
(86) PCT No.: PCT/US2022/018291

§ 371 Date: Sep. 1, 2023

Related U.S. Application Data
(60) Provisional application No. 63/155,378, filed on Mar. 2, 2021.

(51) Int. Cl. A61K 31/497; A61P 1/00

(52) U.S. Cl. CPC A61K 31/497; A61P 1/00

(57) ABSTRACT

Provided are methods of treatment of abdominal pain due to IBS, comprising administering to an individual in need thereof (1aS,5aS)-2-(4-Oxy-pyrazin-2-yl)-1a,2,5,5a-tetrahydro-1H-2,3-diaza-cyclopropa[a]pentalene-4-carboxylic acid ((S)-1-Hydroxymethyl-2,2-dimethyl-propyl)-amide or a pharmaceutically acceptable salt or crystal form thereof.

Irritable bowel syndrome (IBS) is a functional gastrointestinal (GI) disease defined according to Rome IV criteria by recurrent abdominal pain associated with defecation or a change in bowel habits (Lacy 2016). Patients with IBS may also experience other abdominal symptoms of cramping, bloating, and abdominal distension and have lower scores in quality of life measures compared to others with chronic diseases (ten Berg 2006). There are 3 main subtypes of IBS: IBS-C (predominant constipation), IBS-D (predominant diarrhea), and IBS-M (mixed bowel habits). Research (Rey de Castro 2015) suggests that the frequency and severity of pain attacks has been shown to be generally similar across subtypes, but with some differences in duration and frequency of IBS symptomatic episodes (Hellstrom 2011, Weinland 2011).

In Western countries, the prevalence of IBS has been estimated at approximately 10-15%, but with considerably greater country and region variability globally (Hungin 2005, Saito 2002, Sperber 2017). IBS accounts for more than 50% of referrals to GI specialists (Jones 2000, Sandler 1984). Although the underlying cause of IBS is unknown, evidence suggests that multiple biological factors, including motility, epithelial hyperpermeability, dysbiosis, inflammation and immune dysfunction, visceral hypersensitivity, epigenetics/genetics, altered brain-gut interactions, and various psychosocial factors (e.g., response to past and present stressors, cognitive status, and coping behaviors) may all contribute to the pathogenesis of this disorder (Drossman 2016, Enck 2016).

Many patients with IBS report abdominal pain as their most severe IBS symptom (Drossman 2009). Attempts to address IBS-related pain with centrally acting pain medications such as opioids, gabapentin, and tricyclic antidepressants has resulted in limited success, primarily due to the safety profile of these medications. Therefore, treatment of pain in this population remains underdeveloped (Camilleri 2018).

Cannabinoid receptors 1 and 2 (CB₁ and CB₂, respectively) play critical roles in pain perception. CB₁/CB₂ agonists have been shown to alleviate acute, chronic inflammatory, postsurgical, cancer, and neuropathic pain in animal models. However, the therapeutic potential of nonselective CB₁/CB₂ agonists is limited by the psychotropic effects resulting from activation of CB₁ located in the brain. In validated rodent models of inflammatory, neuropathic, and postoperative pain, CB₂-selective agonists have been shown to exhibit analgesic, anti-hyperalgesic, and anti-allodynic activity without psychotropic consequences (Guindon 2008).

Certain CB₂ receptor agonists are useful in the treatment of pain. There is a need in the art for developing methods of using CB₂ receptor agonists in safe and effective therapies and to reduce the risk of adverse events. The methods described herein satisfy this need and provide related advantages as well.

### SUMMARY

According to some embodiments, a method for treating or alleviating abdominal pain due to irritable bowel syndrome (IBS) includes administration of a therapeutically effective amount of (1aS,5a5)-2-(4-oxy-pyrazin-2-yl)-1a,2,5,5a-tetrahydro-1H-2,3-diaza-cyclopropa[a]pentalene-4-carboxylic acid ((S)-1-hydroxymethyl-2,2-dimethyl-propyl)-amide, (Compound A) having the structure:

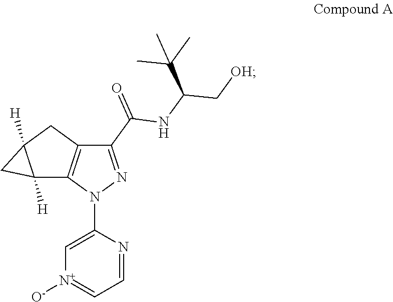 [SMILES: CC(C)(C)[C@@H](CO)NC(=O)c1nn(-c2c[n+]([O-])ccn2)c2c1C[C@@H]1C[C@H]21]

or a pharmaceutically acceptable salt or crystal form thereof to a patient in need thereof. In some embodiments the IBS is selected from irritable bowel syndrome with predominant diarrhea (IBS-D) and irritable bowel syndrome with predominant constipation (IBS-C). In some embodiments, the patient is administered a dose from 10 mg to 500 mg of Compound A. In some embodiments, the patient is administered a dose of 25 mg, 50 mg, 75 mg, 100 mg, 150 mg, 200 mg, 250 mg, 300 mg, 350 mg, 400 mg, 450 mg, or 500 mg of Compound A. In some embodiments, the patient is administered a dose of 50 mg, 100 mg, or 200 mg of Compound A. In some embodiments, the patient is administered a dose of 25 mg, 50 mg, 75 mg, or 100 mg of Compound A. In some embodiments, the Compound A is administered once, twice, or three times per day. In some embodiments, the method is therapeutically effective to improve the average abdominal pain score (AAPS) in the patient from baseline. According to some embodiments, the method is therapeutically effective to achieve at least a 30% improvement in AAPS from baseline.

According to some embodiments, a method for treating or alleviating moderate to severe abdominal pain due to irritable bowel syndrome includes administration of a therapeutically effective amount of Compound A or a pharmaceutically acceptable salt or crystal form thereof, to a patient in need thereof. In some embodiments the IBS is selected from irritable bowel syndrome with predominant diarrhea (IBS-D) and irritable bowel syndrome with predominant constipation (IBS-C). In some embodiments, the patient is administered a dose from 10 mg to 500 mg of Compound A. In some embodiments, the patient is administered a dose of 25 mg, 50 mg, 75 mg, or 100 mg of Compound A. In some embodiments, the patient is administered a dose of, or a dose of about, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, or 500 mg of Compound A. In some embodiments, the patient is administered a dose of 50 mg, 100 mg, or 200 mg of Compound A. In some embodiments, the Compound A is administered once, twice, or three times per day. According to some embodiments, the Compound A is administered in an anhydrous, non-solvated crystalline form. In some embodiments, the method is therapeutically effective to improve the average abdominal pain score (AAPS) in the patient from baseline. According to some embodiments, the method is therapeutically effective to achieve at least a 30% improvement in AAPS from baseline. In some embodiments, the patient has a baseline AAPS score equal to or greater than 5. In some embodiments, the patient has a baseline AAPS score equal to or greater than 6. In some embodiments, the patient has a baseline AAPS score equal to or greater than 6.5. In some embodiments, the patient has a baseline AAPS score equal to or greater than 7. In some embodiments, the dose of Compound A is administered three times per day.

According to some embodiments, a method of reducing the average abdominal pain score (AAPS) in an individual having an average abdominal pain score (AAPS) of five or greater, includes administering a therapeutically effective amount of Compound A or a pharmaceutically acceptable salt or crystal form thereof, to a patient in need thereof. According to some embodiments, a method of reducing the average abdominal pain score (AAPS) in an individual having an average abdominal pain score (AAPS) of six or greater, includes administering a therapeutically effective amount of Compound A or a pharmaceutically acceptable salt or crystal form thereof, to a patient in need thereof. In some embodiments, the patient is administered a dose from 10 mg to 500 mg of Compound A. In some embodiments, the patient is administered a dose of 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, or 500 mg of Compound A. In some embodiments, the patient is administered a dose of 50 mg, 100 mg, or 200 mg of Compound A. In some embodiments, the patient is administered a dose of 25 mg, 50 mg, 75 mg, or 100 mg of Compound A. In some embodiments, the Compound A is administered once, twice, or three times per day. According to some embodiments, the Compound A is administered in an anhydrous, non-solvated crystalline form. According to some embodiments, the method is therapeutically effective to achieve at least a 30% improvement in AAPS from baseline. In some embodiments, the patient has a baseline AAPS score equal to or greater than 6.5. In some embodiments, the patient has a baseline AAPS score equal to or greater than 7. In some embodiments, the dose of Compound A is administered three times per day. According to certain embodiments, the patient suffers no serious adverse events.

According to some embodiments, a method of reducing the average abdominal pain score (AAPS) in an individual having an average abdominal pain score (AAPS) of 6.5 or greater, includes orally administering a therapeutically effective amount of Compound A or a pharmaceutically acceptable salt or crystal form thereof, in an amount of 50 mg, at a frequency of three times a day, to a patient in need thereof. In an embodiment, the Compound A or a pharmaceutically acceptable salt or crystal form thereof is administered to the patient for 12 weeks, and the method is therapeutically effective to achieve a ≥30% improvement in the patient's AAPS for at least 6 of the 12 weeks.

According to some embodiments, a method for treating or alleviating moderate to severe abdominal pain due to irritable bowel syndrome includes orally administering a therapeutically effective amount of Compound A or a pharmaceutically acceptable salt or crystal form thereof, in an amount of 50 mg, at a frequency of three times a day, to a patient in need thereof. In an embodiment, the Compound A or a pharmaceutically acceptable salt or crystal form thereof is administered to the patient for 12 weeks, and the method is therapeutically effective to achieve a ≥30% improvement in the patient's AAPS for at least 6 of the 12 weeks.

According to some embodiments, a method of reducing the average abdominal pain score (AAPS) in an individual having an average abdominal pain score (AAPS) of 6.5 or greater, includes orally administering a therapeutically effective amount of Compound A or a pharmaceutically acceptable salt or crystal form thereof, in an amount of 75 mg, at a frequency of three times a day, to a patient in need thereof. In an embodiment, the Compound A or a pharmaceutically acceptable salt or crystal form thereof is administered to the patient for 12 weeks, and the method is therapeutically effective to achieve a ≥30% improvement in the patient's AAPS for at least 6 of the 12 weeks.

According to some embodiments, a method for treating or alleviating moderate to severe abdominal pain due to irritable bowel syndrome includes orally administering a therapeutically effective amount of Compound A or a pharmaceutically acceptable salt or crystal form thereof, in an amount of 75 mg, at a frequency of three times a day, to a patient in need thereof. In an embodiment, the Compound A or a pharmaceutically acceptable salt or crystal form thereof is administered to the patient for 12 weeks, and the method is therapeutically effective to achieve a ≥30% improvement in the patient's AAPS for at least 6 of the 12 weeks.

According to some embodiments, a method of reducing the average abdominal pain score (AAPS) in an individual having an average abdominal pain score (AAPS) of 6.5 or greater, includes orally administering a therapeutically effective amount of Compound A or a pharmaceutically acceptable salt or crystal form thereof, in an amount of 100 mg, at a frequency of three times a day, to a patient in need thereof. In an embodiment, the Compound A or a pharmaceutically acceptable salt or crystal form thereof is administered to the patient for 12 weeks, and the method is therapeutically effective to achieve a ≥30% improvement in the patient's AAPS for at least 6 of the 12 weeks.

According to some embodiments, a method for treating or alleviating moderate to severe abdominal pain due to irritable bowel syndrome includes orally administering a therapeutically effective amount of Compound A or a pharmaceutically acceptable salt or crystal form thereof, in an amount of 100 mg, at a frequency of three times a day, to a patient in need thereof. In an embodiment, the Compound A or a pharmaceutically acceptable salt or crystal form thereof is administered to the patient for 12 weeks, and the method is therapeutically effective to achieve a ≥30% improvement in the patient's AAPS for at least 6 of the 12 weeks.

According to some embodiments, a method of reducing the average abdominal pain score (AAPS) in an individual having an average abdominal pain score (AAPS) of 6.5 or greater, includes orally administering a therapeutically effective amount of Compound A or a pharmaceutically acceptable salt or crystal form thereof, in an amount of 200 mg, at a frequency of three times a day, to a patient in need thereof. In an embodiment, the Compound A or a pharmaceutically acceptable salt or crystal form thereof is administered to the patient for 12 weeks, and the method is therapeutically effective to achieve a ≥30% improvement in the patient's AAPS for at least 6 of the 12 weeks.

According to some embodiments, a method for treating or alleviating moderate to severe abdominal pain due to irritable bowel syndrome includes orally administering a therapeutically effective amount of Compound A or a pharmaceutically acceptable salt or crystal form thereof, in an amount of 200 mg, at a frequency of three times a day, to a patient in need thereof. In an embodiment, the Compound A or a pharmaceutically acceptable salt or crystal form thereof is administered to the patient for 12 weeks, and the method is therapeutically effective to achieve a ≥30% improvement in the patient's AAPS for at least 6 of the 12 weeks. According to some embodiments, a method for treating or alleviating abdominal pain due to irritable bowel syndrome with predominant constipation (IBS-C) in a patient with a baseline AAPS score equal to or greater than 6, includes administration of a therapeutically effective amount of (1aS,5aS)-2-(4-oxy-pyrazin-2-yl)-1a,2,5,5a-tetrahydro-1H-2,3-diaza-cyclopropa[a]pentalene-4-carboxylic acid ((S)-1-hydroxymethyl-2,2-dimethyl-propyl)-amide, (Compound A) or a pharmaceutically acceptable salt or crystal form thereof to a patient in need thereof. In an embodiment, the patient has a baseline AAPS score equal to or greater than 6.5. In an embodiment, the patient has a baseline AAPS score equal to or greater than 7.

### DETAILED DESCRIPTION

This disclosure provides methods of treating a human subject in need of treatment with a selective CB₂ receptor agonist. Specifically, this disclosure provides methods of treating or alleviating abdominal pain due to irritable bowel syndrome comprising administration of a therapeutically effective amount of a selective CB₂ receptor agonist.

### Selective CB₂ Receptor Agonists

Compounds that interact with and stimulate the CB₂ receptor (which may also be referred to herein as “CB₂ receptor agonists” or “CB₂ agonists”) have utility for the treatment of CB₂ receptor-mediated disorders. In certain embodiments, the agonist compound is selective for the CB₂ receptor relative to the CB₁ receptor. In some embodiments, the agonist compound is selective for the human CB₂ receptor relative to the human CB₁ receptor.

Non-limiting examples of CB₂ receptor agonist compounds are disclosed in PCT patent publications WO2011/025541, WO2012/116276, WO2012/116278, WO2012/116277, and WO2012/116279, and U.S. provisional patent application 62/084,165 (WO2016/085941), which are each incorporated herein by reference in their entirety. For example, CB₂ receptor agonist compounds include Compounds 493, 696, 699, 700, 704, 765, and 820 disclosed in WO2011/025541. These compounds can be prepared as disclosed in WO2011/025541.

In another embodiment, the CB₂ receptor agonist is the compound (1aS,5aS)-2-(4-Oxy-pyrazin-2-yl)-1a,2,5,5a-tetrahydro-1H-2,3-diaza-cyclopropa[a]pentalene-4-carboxylic acid ((S)-1-Hydroxymethyl-2,2-dimethyl-propyl)-amide (“Compound A”) with the chemical structure shown below:

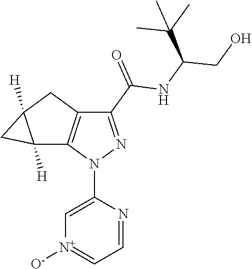 [SMILES: CC(C)(C)[C@@H](CO)NC(=O)c1nn(-c2c[n+]([O-])ccn2)c2c1C[C@@H]1C[C@H]21]

This compound can be prepared as described in Example 1.80 of WO2011/025541 and is referred to as Compound 699 in this PCT publication. This compound is also referred to herein as APD371 or olorinab.

As used herein “APD371” refers to (1aS,5aS)-2-(4-Oxy-pyrazin-2-yl)-1a,2,5,5a-tetrahydro-1H-2,3-diaza-cyclopropa[a]pentalene-4-carboxylic acid ((5)-1-Hydroxymethyl-2,2-dimethyl-propyl)-amide, having the chemical structure shown above (i.e., Compound A), and all chemical and physical forms thereof, including but not limited to, APD371, amorphous forms of APD371, crystalline forms of APD371, crystalline polymorphs of APD371, crystalline habits of APD371, solvates of APD371, amorphous forms of solvates of APD371, crystalline forms of solvates of APD371, crystalline polymorphs of solvates of APD371, crystalline habits of solvates of APD371, hydrates of APD371, amorphous forms of hydrates of APD371, crystalline forms of hydrates of APD371, crystalline polymorphs of hydrates of APD371, crystalline habits of hydrates of APD371, pharmaceutically acceptable salts of APD371, amorphous forms of pharmaceutically acceptable salts of APD371, crystalline forms of pharmaceutically acceptable salts of APD371, crystalline polymorphs of pharmaceutically acceptable salts of APD371, crystalline habits of pharmaceutically acceptable salts of APD371, solvates of pharmaceutically acceptable salts of APD371, amorphous forms of solvates of pharmaceutically acceptable salts of APD371, crystalline forms of solvates of pharmaceutically acceptable salts of APD371, crystalline polymorphs of solvates of pharmaceutically acceptable salts of APD371, crystalline habits of solvates of pharmaceutically acceptable salts of APD371, hydrates of pharmaceutically acceptable salts of APD371, amorphous forms of hydrates of pharmaceutically acceptable salts of APD371, crystalline forms of hydrates of pharmaceutically acceptable salts of APD371, crystalline polymorphs of hydrates of pharmaceutically acceptable salts of APD371, crystalline habits of hydrates of pharmaceutically acceptable salts of APD371, and isotopic enrichment (e.g., deuterium) analogues of any of the above.

APD371 has demonstrated sustained efficacy in models of osteoarthritis pain, paclitaxel-induced neuropathic pain, and painful peripheral diabetic neuropathy. This compound also demonstrates >1,000-fold selectivity for the human CB₂ receptor versus the human CB₁ receptor. This compound also demonstrated a high receptor internalization efficacy for rat and human CB₂ receptors relative to CP55,940 (105% and 96%, respectively). In an osteoarthritis pain model, this compound maintained in vivo efficacy for four hours following dosing, despite rapidly declining plasma concentrations. Methods of treating pain, including visceral pain with APD371 are described in US patent application publication no. 2020-0078358, which is herein incorporated by reference in its entirety.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate.

Compounds of the present invention or a solvate, hydrate or physiologically functional derivative thereof can be used as active ingredients in pharmaceutical compositions, specifically as cannabinoid receptor modulators. By the term “active ingredient” is defined in the context of a “pharmaceutical composition” and is intended to mean a component of a pharmaceutical composition that provides the primary pharmacological effect, as opposed to an “inactive ingredient” which would generally be recognized as providing no pharmaceutical benefit.

Formulations may be prepared by any suitable method, typically by uniformly mixing the active compound(s) with liquids or finely divided solid carriers, or both, in the required proportions and then, if necessary, forming the resulting mixture into a desired shape.

Conventional excipients, such as binding agents, fillers, acceptable wetting agents, tableting lubricants and disintegrants may be used in tablets and capsules for oral administration. Liquid preparations for oral administration may be in the form of solutions, emulsions, aqueous or oily suspensions and syrups. Alternatively, the oral preparations may be in the form of dry powder that can be reconstituted with water or another suitable liquid vehicle before use. Additional additives such as suspending or emulsifying agents, non-aqueous vehicles (including edible oils), preservatives and flavorings and colorants may be added to the liquid preparations. Parenteral dosage forms may be prepared by dissolving the compound of the invention in a suitable liquid vehicle and filter sterilizing the solution before filling and sealing an appropriate vial or ampule. These are just a few examples of the many appropriate methods well known in the art for preparing dosage forms.

A compound of the present invention can be formulated into pharmaceutical compositions using techniques well known to those in the art. Suitable pharmaceutically-acceptable carriers, outside those mentioned herein, are known in the art; for example, see Remington, The Science and Practice of Pharmacy, 20^{th} Edition, 2000, Lippincott Williams & Wilkins, (Editors: Gennaro et al.).

While it is possible that, for use in the prophylaxis or treatment, a compound of the invention may, in an alternative use, be administered as a raw or pure chemical, it is preferable however to present the compound or active ingredient as a pharmaceutical formulation or composition further comprising a pharmaceutically acceptable carrier.

The compounds of the invention, together with a conventional adjuvant, carrier, or diluent, may be placed into the form of pharmaceutical formulations and unit dosages thereof and in such form may be employed as solids, such as tablets or filled capsules, or liquids such as solutions, suspensions, emulsions, elixirs, gels or capsules filled with the same. Such pharmaceutical compositions and unit dosage forms thereof may comprise conventional ingredients in conventional proportions, with or without additional active compounds or principles and such unit dosage forms may contain any suitable effective amount of the active ingredient commensurate with the intended daily dosage range to be employed.

The dose when using the compounds of the present invention can vary within wide limits and as is customary and is known to the physician, it is to be tailored to the individual conditions in each individual case. It depends, for example, on the nature and severity of the illness to be treated, on the condition of the patient, on the compound employed or on whether an acute or chronic disease state is treated or prophylaxis conducted or on whether further active compounds are administered in addition to the compounds of the present invention. Some non-limiting preferred dosages for inclusion in the compositions and methods of the present disclosure include about: 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85, mg, 90 mg, 95 mg, 100 mg, 110 mg, 120 mg, 125 mg, 130 mg, 140 mg, 150 mg, 160 mg, 170 mg, 175 mg, 180 mg, 190 mg, 200 mg, 210 mg, 220 mg, 225 mg, 230 mg, 240 mg, 250 mg, 260 mg, 275 mg, 280 mg, 290 mg, 300 mg, 325 mg, 350 mg, 375 mg, 400 mg, 425 mg, and 450 mg. Multiple doses may be administered during the day, especially when relatively large amounts are deemed to be needed, for example 2, 3, or 4 doses. Depending on the individual and as deemed appropriate from the patient's physician or caregiver it may be necessary to deviate upward or downward from the doses described herein. Adjustment to the dosages of compounds of the present invention are disclosed, for example, in US Patent Application Publication No. 2019-0160058, which is herein incorporated by reference in its entirety.

In some embodiments, about 50, 55, 60, 65, 70, 75, 80, 85, 90, 95, 100, 105, 110, 115, 120, 125, 130, 135, 140, 145, or 150 mg of Compound A is administered three times per day.

In some embodiments, about 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, or 225 mg of Compound A is administered two times per day.

In some embodiments, about 150 mg, 160 mg, 170 mg, 175 mg, 180 mg, 190 mg, 200 mg, 210 mg, 220 mg, 225 mg, 230 mg, 240 mg, 250 mg, 260 mg, 270 mg, 275 mg, 280 mg, 290 mg, 300 mg, 325 mg, 350 mg, 375 mg, 400 mg, 425 mg, or 450 mg of Compound A is administered per day.

According to some embodiments, the present disclosure provides a method for treating or alleviating abdominal pain in a patient in need of such treatment, comprising administering to the patient a therapeutically effective amount of Compound A and pharmaceutically acceptable salts and N-oxides thereof. In some embodiments, the abdominal pain treated is pain is due to Irritable Bowel Syndrome (IBS). In some embodiments, the abdominal pain treated is pain is due to Irritable Bowel Syndrome with predominant constipation (IBS-C). In some embodiments, the abdominal pain treated is pain is due to Irritable Bowel Syndrome with predominant diarrhea (IBS-D). In some embodiments, the abdominal pain treated is pain is due to Irritable Bowel Syndrome with mixed bowel habits (IBS-M). In some embodiments, the abdominal pain treated is pain is due to unsubtyped Irritable Bowel Syndrome (IBS-U).

In some embodiments, the abdominal pain can be described as visceral pain. Visceral pain generally is caused by the activation of pain receptors in the chest, abdomen, or pelvic areas, and can be caused by injury or disease states involving the internal organs, such as the stomach, kidney, gallbladder, urinary bladder, and intestines. Visceral pain can also be caused by problems with abdominal muscles and the abdominal wall, such as spasm. Visceral pain is distinct from somatic pain, which is caused by the activation of pain receptors in either the body surface or musculoskeletal tissues (for example, postsurgical pain from a surgical incision), and from neuropathic pain, which is caused by injury or malfunction to the spinal cord and peripheral nerves. In some embodiments, the visceral abdominal pain does not arise from or relate to inflammatory bowel disease. In some embodiments, the visceral abdominal pain does not arise from or relate to Crohn's disease.

Treatment of pain can be assessed by scales and measurements known by those of skill in the art. In some embodiments, treatment of pain is assessed on the Average Abdominal Pain Score (AAPS). The Abdominal Pain Score (APS) is a single-question, 11-point numeric rating scale in which 0 represents no abdominal pain and 10 represents the worst possible abdominal pain. In some embodiments, moderate to severe abdominal pain includes an AAPS equal to or greater than 5. In some embodiments moderate to severe abdominal pain includes an AAPS equal to or greater than 6. In some embodiments moderate to severe abdominal pain includes an AAPS equal to or greater than 6.5. In some embodiments moderate to severe abdominal pain includes an AAPS equal to or greater than 7. In some embodiments, a method for treating or alleviating moderate to severe abdominal pain due to IBS in a patient in need of such treatment, comprises administering to the patient a therapeutically effective amount of Compound A and pharmaceutically acceptable salts and N-oxides thereof. In some embodiments, a method for treating or alleviating severe abdominal pain due to IBS in a patient in need of such treatment, comprises administering to the patient a therapeutically effective amount of Compound A and pharmaceutically acceptable salts and N-oxides thereof. In some embodiments, a method for treating or alleviating moderate to severe abdominal pain due to IBS-C in a patient in need of such treatment, comprises administering to the patient a therapeutically effective amount of Compound A and pharmaceutically acceptable salts and N-oxides thereof. In some embodiments, a method for treating or alleviating severe abdominal pain due to IBS-C in a patient in need of such treatment, comprises administering to the patient a therapeutically effective amount of Compound A and pharmaceutically acceptable salts and N-oxides thereof. In some embodiments, a method for treating or alleviating moderate to severe abdominal pain due to IBS-D in a patient in need of such treatment, comprises administering to the patient a therapeutically effective amount of Compound A and pharmaceutically acceptable salts and N-oxides thereof. In some embodiments, a method for treating or alleviating severe abdominal pain due to IBS-D in a patient in need of such treatment, comprises administering to the patient a therapeutically effective amount of Compound A and pharmaceutically acceptable salts and N-oxides thereof. In some embodiments, moderate to severe abdominal pain comprises an AAPS score equal to or greater than 6. In some embodiments, moderate to severe pain comprises an AAPS score equal to or greater than 7. In some embodiments, severe pain comprises an AAPS score equal to or greater than 6. In some embodiments, severe pain comprises an AAPS score equal to or greater than 7.

In some embodiments, the method is therapeutically effective to reduce a patient's AAPS score by 30% from the patient's baseline AAPS score. In some embodiments, the method is therapeutically effective to reduce a patient's AAPS score by 40% from the patient's baseline AAPS score. In some embodiments, the method is therapeutically effective to reduce a patient's AAPS score by 50% from the patient's baseline AAPS score. In some embodiments, the method is therapeutically effective to achieve ≥30% improvement in AAPS from baseline for at least 6 of 12 weeks.

In some embodiments, Compound A or a pharmaceutically acceptable salt or N-oxide thereof is administered to a patient with a low or minimal occurrence of adverse events. An adverse event is an untoward medical occurrence that is associated with treatment with Compound A or a pharmaceutically acceptable salt or N-oxide thereof. In one embodiment, an adverse event is selected from: headache, nausea, vomiting, back pain, and menstrual disorder. In some embodiments, Compound A, or a pharmaceutically acceptable salt or N-oxide thereof, is administered without causing a serious adverse event.

In some embodiments, the individual is administered Compound A or a pharmaceutically acceptable salt or N-oxide thereof for at least one month, such as one month, two months, three months, four months, etc. In some embodiments, the individual is administered Compound 1 or a pharmaceutically acceptable salt thereof for least one week, such as one week, two weeks, three weeks, four weeks, five weeks, six weeks, seven weeks, eight weeks, nine weeks, ten weeks, eleven weeks, twelve weeks, thirteen weeks, fourteen weeks, fifteen weeks, sixteen weeks, etc. In some embodiments, the period is indefinite, e.g., chronic administration.

### EXAMPLES

### Example 1: Clinical Trial

A Phase 2, multi-center, randomized, double blind, placebo-controlled parallel group study was conducted to evaluate the safety, tolerability, and efficacy of olorinab (Compound A) in subjects with irritable bowel syndrome experiencing abdominal pain. Specifically, this study was designed to assess the efficacy, safety, and tolerability of Compound A in the treatment of abdominal pain in subjects with IBS with predominant constipation (IBS-C) or IBS with predominant diarrhea (IBS-D) who are not on concomitant treatment for IBS. The study included a screening period (up to four weeks for subjects who consent to colonic biopsy, up to two weeks for all other subjects), a run-in period (two weeks), a randomized main study treatment period (12 weeks), and a post treatment follow up period (two weeks), totaling 16-20 weeks. After the run-in period, eligible subjects were equally randomized into 1 of 4 treatment groups (Compound A 10 mg, 25 mg, or 50 mg (tablet or powder in capsule) three times per day [tid] or placebo tid). Randomization was stratified by sex and IBS subtype. The number of subjects enrolled with IBS-C were approximately equal to the number of subjects enrolled with IBS-D. Subjects were screened until approximately 60 subjects have been randomized per study group, for a total of approximately 240 subjects.

The study treatment (tablet or capsule) was administrated orally with water. Subjects were instructed to administer their study medication tid (approximately every 8 hours) during the subject's normal wakeful hours.

### Inclusion Criteria:

1. Male and female subjects ≥18 and ≤70 years of age at Visit 1 (Screening)
2. Body mass index ≥18.0 and ≤40.0 kg/m² at Visit 1 (Screening)
3. Clinical diagnosis of IBS-C or IBS-D according to Rome IV criteria at Visit 1 (Screening)
4. Per the Rome IV diagnostic algorithm for IBS, subjects 50 years of age and over had one of the following with a result that rules out causes of abdominal pain other than IBS:
   a. Colonoscopy (within 10 years of Visit 1 [Screening])
   b. Flexible sigmoidoscopy and double contrast barium enema (within 5 years of Visit 1 [Screening])
   c. Computed tomography (CT) colonography (within 5 years of Visit 1 [Screening])
5. Subjects with a family history in a first degree relative of colorectal cancer or inflammatory bowel disease or recent (within 6 months of Visit 1 [Screening]) or ongoing alarm features (unexplained weight loss, nocturnal symptoms, blood mixed with stool) had a diagnostic colonoscopy prior to Screening (Visit 1) and after the onset of alarm features (for subjects with alarm features) to exclude non-IBS conditions per the Rome IV diagnostic algorithm for IBS.
6. If treated with any of the following medications, dosing was stable for 90 days prior to Screening and the subject agreed to maintain the same dose and frequency of medication throughout the study:
   a. Tricyclic antidepressants, tetracyclic antidepressants, selective serotonin reuptake inhibitors (SSRIs), serotonin and norepinephrine reuptake inhibitor (SNRIs), or anticonvulsants (e.g., pregabalin or gabapentin) for conditions other than IBS pain
   b. Benzodiazepines or non-benzodiazepine hypnotics, administered at bedtime for conditions other than IBS pain
7. If treated with any of the following medications, dosing (or approximate frequency of ‘as needed’ use) was stable for at least 30 days prior to Visit 1 (Screening) and the subject agreed to maintain the stable dose (or approximate frequency of ‘as needed’ use) of medication throughout the study:
   a. Probiotics
   b. Bulk laxatives, fiber, and stool softeners
   c. Bismuth subsalicylate
8. Able to understand and willing to participate in the study
9. Capable of giving signed informed consent, which includes compliance with the requirements and restrictions listed in the informed consent form (ICF) and in the study protocol
   At the End of the Run-In Period, ONLY Subjects Meeting the Following Electronic Diary (eDiary) Criteria and all Other Inclusion Criteria were Eligible to Progress to Randomization:
10. Minimum of 50% of reported days of the run-in period with abdominal pain >0 and AAPS≥4 throughout the run-in period
11. Adequate compliance with entry of daily eDiary completed for ≥80% of days of the run-in period (Visit 2 to Visit 3 [Day 1])
12. Subject did not use any rescue medication during the run-in period

### Key Exclusion Criteria:

Subjects were excluded from the study if they met any of the following key exclusion criteria.
1. Diagnosis of IBS with mixed bowel habits (IBS-M) or unsubtyped IBS (IBS-U)
2. Clinically relevant changes in dietary, lifestyle, or exercise regimen within 30 days prior to Visit 1 (Screening) that may confound efficacy assessments in the clinical judgment of the Investigator (or designee)
3. Any colonic or major abdominal surgery (e.g., bariatric surgery [including gastric banding], stomach surgery, small/large bowel surgery, or abdominal large vessel surgery). History of cholecystectomy was exclusionary for subjects with IBS-D. For subjects with IBS-C, a history of cholecystectomy more than 6 months prior to Visit 1 (Screening) was allowed. Procedures such as appendectomy, hysterectomy, caesarean section, or polypectomy were allowed as long as they occurred at least 3 months prior to Visit 1 (Screening).
4. History of colorectal cancer, inflammatory bowel disease (IBD), diverticulitis, ischemic colitis, microscopic colitis, bile acid diarrhea, or celiac disease. If a subject with IBS-D has not previously been tested for celiac disease, then the absence of celiac disease was confirmed by testing immunoglobulin A (IgA) anti-tissue transglutaminase (tTG) with total IgA levels; if the subject had IgA deficiency, then IgA/immunoglobulin G (IgG) anti-deamidated gliadin peptide antibody (DGP) tests were performed with a result that rules out celiac disease.
5. History of organic abnormalities of the gastrointestinal (GI) tract, intestinal obstruction, stricture, toxic megacolon, GI perforation, fecal impaction, or impaired intestinal circulation (e.g., aortoiliac disease)
6. Other GI diseases such as peptic ulceration, functional dyspepsia, GI bleeding, or GI inflammatory disease (e.g., esophagitis, gastritis, or duodenitis) within 6 months prior to Visit 1 (Screening). The following conditions did not exclude a subject: acute gastritis that resolved without complication, and gastroesophageal reflux disease (GERD).
7. Use of any of the following medications within 30 days prior to Visit 1 (Screening):
   a. Opioids
   b. The following were excluded if prescribed for IBS pain: tricyclic antidepressants, tetracyclic antidepressants (e.g., mirtazapine), SNRIs, anticonvulsants (e.g., pregabalin or gabapentin)
   c. Medical or recreational marijuana, tetrahydrocannabinol (THC), cannabidiol (CBD), synthetic cannabinoids, or other cannabis derivatives for any indication
   d. Benzodiazepines, or non-benzodiazepine hypnotics, unless administered at bedtime for conditions other than IBS pain
8. Prior (within 14 days of Visit 1 [Screening]) or anticipated concomitant medication for IBS including, but not limited to, abdominal pain medications, antibiotics, anticholinergics, antidiarrheals, antiflatulence agents, antispasmodics, chloride channel activators, bile acid sequestrants, cholinomimetics, 5-HT₃ antagonists, 5-HT₄ agonists, guanylate cyclase C agonists, opioid agonists or antagonists, osmotic laxatives, sodium-proton exchanger NHE3 inhibitors, and stimulant laxatives. NOTE: Use of OTC fiber, bulk laxatives, stool softeners, and bismuth subsalicylate were permitted provided doses were stable for at least 30 days prior to Visit 1 (Screening) and the subject agreed to maintain stable doses (within ±20% total daily dose) or approximate frequency of ‘as needed’ use of medication throughout study participation.
9. Prior (within 30 days of Visit 1 [Screening]) or anticipated concomitant use of GI antibiotics

### Endpoints

### Main Study

### Primary

Change in average abdominal pain score (AAPS) from baseline to week 12
Adverse events (AEs) and clinically relevant changes in vital signs and clinical laboratory results

### Secondary

The proportion of subjects achieving a ≥30% improvement in AAPS from baseline to week 12
The proportion of subjects achieving a ≥30% improvement in AAPS from baseline for at least 6 of the 12 weeks during the main study treatment period
Percent change in AAPS from baseline to week 12
Change in number of pain free days per week from baseline to week 12
PK parameters including, but not limited to, observed maximum concentration (Cₘₐₓ), time of observed maximum (peak) concentration after drug administration (tₘₐₓ), and observed trough (pre-dose) concentration (C_{trough})

### Results

Surprisingly, a clinical meaningful and statistically significant improvement in AAPS was observed at week 12 in patients treated with Compound A 50 mg in the subgroup of subjects with moderate to severe abdominal pain at baseline. Specifically, the method was effective for participants with a baseline AAPS greater than or equal to 6.5, representing those with moderate to severe pain. This group accounted for approximately 50% of the study population. This population showed a clinically meaningful and statistically significant (p=0.01) reduction in AAPS of 1.64 points compared to placebo, and a reduction of 3.93 points from baseline in the 50 mg group at week 12.

Compound A was generally safe and well tolerated in the study. Discontinuation rates and adverse events were similar to placebo, notably with no worsening of bowel habits and no treatment interruptions. There were no serious adverse events in the study.

### Example 2: Clinical Trial for Abdominal Pain

A Phase 2, placebo controlled, parallel group study will be conducted to evaluate the efficacy and safety of olorinab (Compound A) 50 mg, 100 mg and 200 mg TID in subjects with IBS-C experiencing moderate to severe abdominal pain.

The study will enroll subjects with IBS-C with a mean pain score (0-10 scale) of at least 5 who would receive either placebo, 50 mg, 100 mg or 200 mg Compound A for 12 weeks, in the absence of other pain medication.

The primary outcome measure for efficacy will be improvement in mean abdominal pain score. The primary outcome measure for safety will be adverse events, clinical labs, ECG, physical examination and vital signs.

Other uses of the disclosed methods will become apparent to those in the art based upon, inter alia, a review of this patent document.

1. A method for treating or alleviating abdominal pain due to irritable bowel syndrome (IBS) comprising administration of a therapeutically effective amount of (1aS,5aS)-2-(4-oxy-pyrazin-2-yl)-1a,2,5,5a-tetrahydro-1H-2,3-diaza-cyclopropa[a]pentalene-4-carboxylic acid ((S)-1-hydroxymethyl-2,2-dimethyl-propyl)-amide, (Compound A) having the structure: 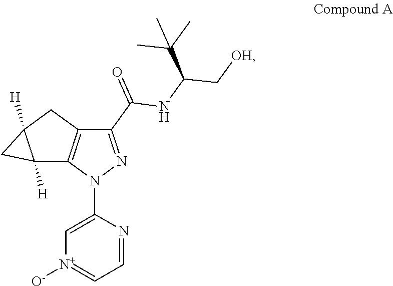 [SMILES: CC(C)(C)[C@@H](CO)NC(=O)c1nn(-c2c[n+]([O-])ccn2)c2c1C[C@@H]1C[C@H]21]
or a pharmaceutically acceptable salt or crystal form thereof to a patient in need thereof.

2. The method of claim 1, wherein the IBS is selected from the group consisting of irritable bowel syndrome with predominant diarrhea (IBS-D) and irritable bowel syndrome with predominant constipation (IBS-C).

3. The method of claim 2, wherein the IBS is IBS-C.

4. The method of claim 2, wherein the IBS is IBS-D.

5. The method of claim 1, wherein the therapeutically effective amount is from about 10 mg to about 500 mg.

6. (canceled)

7. The method of claim 1, wherein the therapeutically effective amount is about 25 mg, about 50 mg, about 75 mg, or about 100 mg.

8. (canceled)

9. The method of claim 1, wherein the Compound A is administered once per day, twice per day, or three times per day.

10. The method of claim 1, wherein Compound A is administered in an anhydrous, non-solvated crystalline form.

11. (canceled)

12. The method of claim 1, wherein the method is therapeutically effective to achieve at least a 30% improvement in an average abdominal pain score (AAPS) from a baseline AAPS measured prior to administration of Compound A or a pharmaceutically acceptable salt or crystal form thereof.

13-24. (canceled)

25. A method of reducing an average abdominal pain score (AAPS) in an individual having an AAPS of six or greater, comprising administering a therapeutically effective amount of (1aS,5aS)-2-(4-oxy-pyrazin-2-yl)-1a,2,5,5a-tetrahydro-1H-2,3-diaza-cyclopropa[a]pentalene-4-carboxylic acid ((S)-1-hydroxymethyl-2,2-dimethyl-propyl)-amide, (Compound A) having the structure: 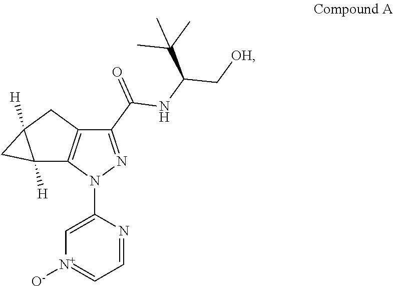 [SMILES: CC(C)(C)[C@@H](CO)NC(=O)c1nn(-c2c[n+]([O-])ccn2)c2c1C[C@@H]1C[C@H]21]
or a pharmaceutically acceptable salt or crystal form thereof to a patient in need thereof.

26. The method of claim 25, wherein the therapeutically effective amount is from about 10 mg to about 500 mg.

27. (canceled)

28. The method of claim 25, wherein the therapeutically effective amount is about 25 mg, about 50 mg, about 75 mg, or about 100 mg.

29. (canceled)

30. The of claim 25, wherein Compound A, or a pharmaceutically acceptable salt or crystal form thereof, is administered once per day, twice per day, or three times per day.

31. The method of claim 25, wherein Compound A, or a pharmaceutically acceptable salt or crystal form thereof, is administered in an anhydrous, non-solvated crystalline form.

32. The method of claim 25, wherein the method is therapeutically effective to achieve at least a 30% improvement in an AAPS from a baseline AAPS measured prior to administration of Compound A, or a pharmaceutically acceptable salt or crystal form thereof.

33. The method of claim 25, wherein the patient has a baseline AAPS equal to or greater than 6.5.

34. The method of claim 25, wherein the patient has a baseline AAPS equal to or greater than 7.

35. The method of claim 25, wherein Compound A, or a pharmaceutically acceptable salt or crystal form thereof, is administered three times per day.

36-39. (canceled)

40. The method of claim 25, wherein Compound A or a pharmaceutically acceptable salt or crystal form thereof is administered to the patient for 12 weeks, and wherein the method is therapeutically effective to achieve a ≥30% improvement in the patient's AAPS for at least 6 of the 12 weeks from a baseline AAPS measured prior to administration of Compound A, or a pharmaceutically acceptable salt or crystal form thereof.

41-55. (canceled)
